(19) United States
(12) Patent Application Publication
(10) Pub. No.: US 2006/0000480 A1
(43) Pub. Date: Jan. 5, 2006

(54) Method of infusing a therapeutic fluid into a patient

(76) Inventor: James B. Broselow, Hickory, NC (US)

Correspondence Address: Adams Evans P.A., 2180 Two Wachovia Center, 301 S. Tryon Street, Charlotte, NC, 28282, US

(21) Appl. No.: 10/882,062

(22) Filed: Jun. 30, 2004

(51) Int. Cl. A61B 19/00

(52) U.S. Cl. 128/898

(57) ABSTRACT

A method of infusing a therapeutic fluid into a patient, including the steps of categorizing a universe of patient weights into a plurality of discrete weight ranges, and correlating the weight ranges to a preselected series of identifiably distinct characteristics. An appropriate amount of a therapeutic substance is determined for each of the plurality of weight ranges, along with normal maintenance fluid requirements each one of the plurality of weight ranges. The amount of administered maintenance fluid is adjusted to compensate for the quantity of therapeutic fluid being administered. The identifiably distinct characteristic correlated with the selected one of the plurality of weight ranges is used to visually correlate the patient with a proper therapeutic fluid dosage and a proper amount of maintenance fluid. The therapeutic fluid and maintenance fluid is infused into the patient in amounts collectively comprising a proper amount of fluid based on the correlated weight range of the patient as indicated by the characteristic.

### TECHNICAL FIELD AND BACKGROUND OF THE INVENTION

This invention relates to a method of infusing a therapeutic fluid into a patient. Drug infusions for pediatric and newborn therapy have always been a difficult and error-prone part of clinical medicine. The difficulty lies in the fact that where most IV medications for children are ordered by a single dose, infusions must be varied or “titrated” according to the clinical situation. While a standard dose is ordered by calculating mg/kg of a child's weight, an infusion is typically ordered in micrograms/kg (of body weight)/minute. In addition, the potency and at times the effects of the medication vary by the level of micrograms/kg/minute. An example is the drug dopamine that raises blood pressure while improving renal blood flow at low doses (typically 1 to 5 micrograms/kg/minute infusion rate) while at higher rates (20 micrograms/kg/minute) raises blood pressure but decreases renal output. In other words, the same drug has different effects on the body at different doses.

A confounding issue regarding this problem is also the fact that many infusions that can be given to adults from a dose of 1 to 20 “mics” without concerns about fluid overload. This is because the amounts of fluid in the standard concentrations allow even the highest doses and infusions rates to given without causing fluid overload. The daily fluid requirements of most adults are much greater than the amount of extra fluid in the infusion and this can easily be compensated for.

This is not the situation, however, in children and especially in neonates whereby the daily fluid requirements may be quite small and the amount of fluid given in standard drips might literally “drown” the child. In addition, infusions that make up over 8-10% of maintenance requirements represent a potential problem as regards to nutrition, electrolytes, etc. For this reason when ordering an infusion, the daily fluid requirement for the child must be taken into consideration as well as the desired drug effect.

The standard way of accomplishing this has been to use a complex series of calculations. For example, if an order for 2 micrograms/kg/minute of dopamine were ordered, the nurse or pharmacist would have to determine based on the patient weight the amount of micrograms of that drug that should be given over an hour, what concentrations of that drug are available and how much fluid it would be administered to the child per hour. Once the correct concentration of the drug is determined, the nurse or pharmacist then determines how many mLs per hour would translate into the appropriate rate to correspond to the order of 2 mics per Kg per minute. Once determined on an hourly basis, the amount is divided by 60 to determine the amount of mLs per minute—the manner in which the standard infusion pump is set at the bedside. Ultimately this order is reduced to a bedside setting such as 6 mLs per hour.

The standard way for clinicians, pharmacists and nurses to communicate this is to always express the drug dose in terms of “mics per Kg per minute.” A typical dialog between a doctor and a nurse on this issue might be to tell a nurse to “start a dopamine drip at 2 mics.” In other instances a doctor might walk into a pediatric ICU room making morning rounds or more importantly might be responding to an emergency page about a sudden deterioration in the patient condition and the first question he might ask is “how many mics of dopamine is he on?

The answer to that question is not obvious. One would either just have to accept the nurse's word that the 6 mLs per hour on the pump represents 2 mics/kg/minute, or one would have to go through the entire recalculation process.

One proposed solution to this problem has been the development of infusion pumps that calculate the correct values at the bedside. To do this one would program into the pump the name of the drug, its concentration, the patient weight, and the intended micrograms/kg/minute. The pump would then automatically be set to give that amount. The device could print a paper strip that showed the process. It could still give the wrong answer if the concentration of the drip being given were programmed incorrectly into the device.

One of the problems with this approach is that it is quite time consuming to enter the data, mistakes can be made if the data is entered incorrectly, and one must spend additional time reviewing all the input data to assure that it has been done correctly, particularly since other health care providers will assume that the value has been correctly determined.

At the present time most of these calculations are done individually and not on an infusion pump. In an effort to simplify this process, the American Heart Association developed a mathematical formula called the “Rule of 6's” by which a simple mathematical process can be performed. In this scenario, the child's weight is used to vary the way the dopamine is formulated so that a different concentration is used for each child. With this approach the mics/KG/Hour always equals the Mls per minute This formula however, does not take into consideration the child's total fluid requirements, and more importantly has been found to be error prone. Because of this the JCHAO organization has decided that this procedure will no longer be acceptable in hospitals for calculating infusions after January 2005.

There are large economic implications for simple approach disclosed in this application, in that there is a significant amount of nursing time tied up in programming infusion devices to give the same information. Despite this there is still room for programming errors, and there is the continuing need for hospitals to buy more expensive “programmable” infusion devices that would not be needed with this simple solution. And even if a programmable pump were being used, the color could always be used as part of a “Failsafe system” that could easily assure at the bedside that the pump had been programmed correctly or that the correct maintenance fluids were being given.

### SUMMARY OF THE INVENTION

Therefore, it is an object of the invention to provide a method of infusing therapeutic fluids into a patient.

It is another object of the invention to provide a method of infusing therapeutic fluids into a neonatal or pediatric patient.

It is another object of the invention to provide a method of infusing therapeutic fluids that predetermines the values necessary to properly determine total fluid requirements without weight, time and dosage concentration calculations.

It is another object of the invention to provide a label that includes concentration-matched, weight specific data with a plurality of weight ranges correlated with mics/kg/minute.

These and other objects of the present invention are achieved in the preferred embodiments disclosed below by providing a system of putting pediatric and neonatal patients into easily identifiable color-coded categories and correlating those categories into a series of medication labels that resolves the problems identified above. The issue of fluid administration is solved since only those labels that allow appropriate amounts are available for that particular patient. In addition, the labels show the normal maintenance fluid requirements for the child, allowing the doctor to adjust the maintenance fluids to compensate for any changes being made in the infusion orders. In addition, these same issues of fluid administration relative to total requirements are addressed on the included “Infusion device window” that correlates patient color, mLs per hour and percent of maintenance fluids to help monitor the process. Multiple infusion devices with these windows can be accessed individually at the bedside and changes could then made, or networked so that the percent of maintenance fluids from the entire system is monitored and controlled. Also, simple maintenance requirements are set by scrolling the percent maintenance window to 100%, or other percentage of maintenance fluids selected, based on the particular clinical situation.

One embodiment of the method of infusing a therapeutic fluid into a patient comprises the steps of categorizing a universe of patient weights into a plurality of discrete weight ranges, and correlating the weight ranges to a preselected series of identifiably distinct colors. An appropriate amount of a therapeutic substance for each of the plurality of weight ranges is then determined. Normal maintenance fluid requirements are determined each one of the plurality of weight ranges, and the amount of administered maintenance fluid is adjusted to compensate for the quantity of therapeutic fluid being administered. The identifiably distinct color are correlated with the selected one of the plurality of weight ranges to visually correlate the patient with a proper therapeutic fluid dosage and a proper amount of maintenance fluid. The therapeutic fluid and maintenance fluid is then infused into the patient in amounts collectively comprising a proper amount of fluid based on the correlated weight range of the patient as indicated by the color.

Another embodiment of the invention includes the preparation of pre-printed sheet of labels that increment from the smallest infant weight range compatible with a given concentration to the largest weight range, including adults. The labels intended for children are preferably distinguished by color, whereas the labels intended for adults are categorized by weight.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 is a schematic showing one embodiment of a system for categorizing pediatric patient weights by color;

FIG. 2 is a schematic showing a system of categorizing pediatric neonatal patients into “Elefants” (large neonates) and “Miceronates” (small neonates) giving two neonatal categories. each category subcategorized into the colors shown in FIG. 1;

FIGS. 3 and 4 illustrate selection of a patient based on weight and identified by an easily distinguishable color;

FIG. 5 is an example of a label to be applied to an infusion pump to provide information regarding the amount of a therapeutic fluid to be administered to a large neonate;

FIG. 6 is an example of a label to be applied to an infusion pump to provide information regarding the amount of a therapeutic fluid to be administered to a small neonatal patient;

FIG. 7 is an example of an infusion bag label for a pediatric patient according to the invention of the type to be applied to the infusion bag by a pharmacist;

FIG. 8 is an example of a infusion bag label on a infusion bag as supplied by a pharmacy or manufacturer where the infusion is to be color-coded with an appropriate color sticker by the nurse or pharmacist at the point of care; and

FIG. 9 is an example of the input control panel of an infusion pump intended for use with the labels and in accordance with the method of the invention.

### DESCRIPTION OF THE PREFERRED EMBODIMENT AND BEST MODE

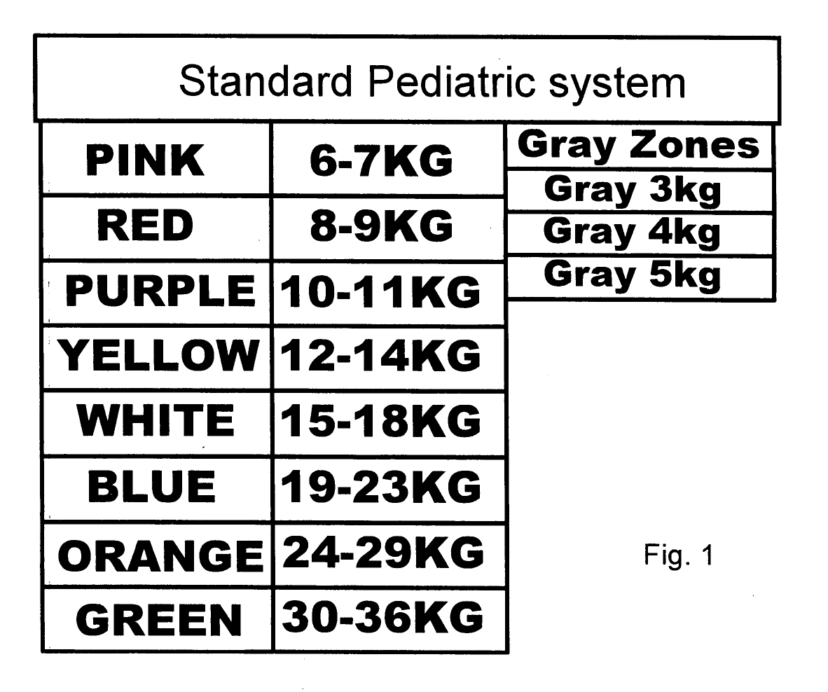

Referring now specifically to the drawings, a method of infusing a therapeutic fluid into a patient according to the present invention is illustrated in the drawings identified above. As is shown in FIG. 1, pediatric patients can be categorized according to body weight. Prior patents by the present applicant have demonstrated that dosing pediatric patients based on length correlated with weight is an efficient and medically desirable means of treating pediatric patients, particularly in an emergency setting. The categorization shown in FIG. 1 is illustrative of a system that determines the physiological effect on pediatric patients according to weight, and takes into account the degree of variation in physiological effect of a medication caused by a degree of change in weight. The categories are then correlated with visually distinct colors. For example, note that the pink, red and purple weight ranges have only a 1 kg spread, and encompass only a total range of 2 kg. In contrast, the green weight range has a 6 kg and a total range of 7 kg.

The “gray” zone represents the 3, 4 and 5 kgs., the color indicating that these are infants, but not neonates. Neonates versus infants of same weight may require different therapeutic approaches since the physiology is different between a 3 kg patient who was born in the last week, versus a 3 kg infant who was born prematurely and is now 2 months old. Also, each kg is subdivided within this zone to allow more accurate dosing.

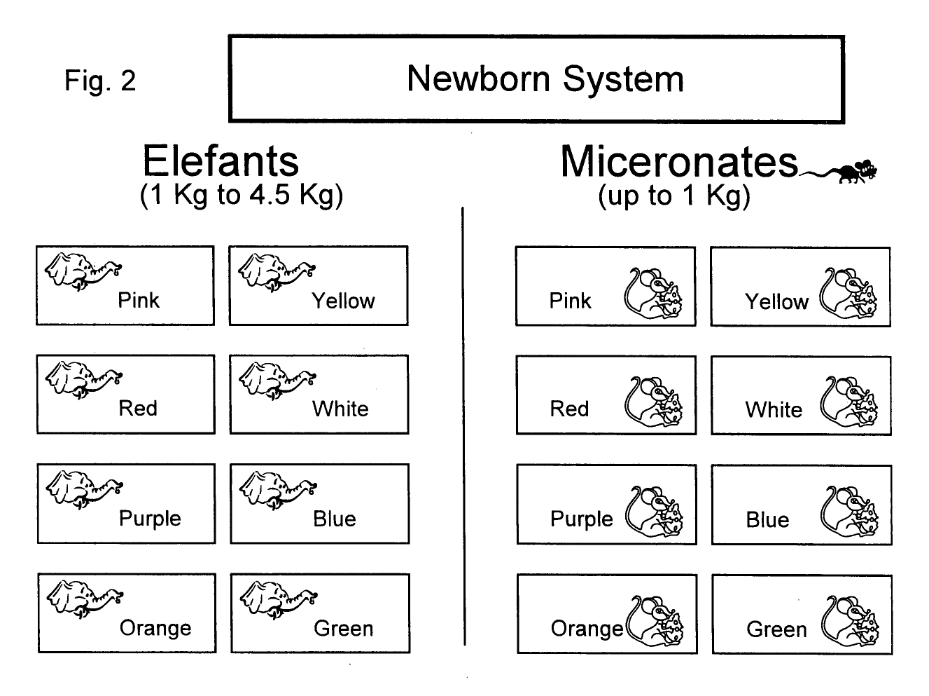

Referring now to FIG. 2, within the “pediatric” patient category are the subcategories of Infant and Neonatal patients, referred to as “Elefants” and “Miceronates”, respectively to indicate relative size. These subcategories are further subcategorized into colors, as with the standard pediatric system shown in FIG. 1.

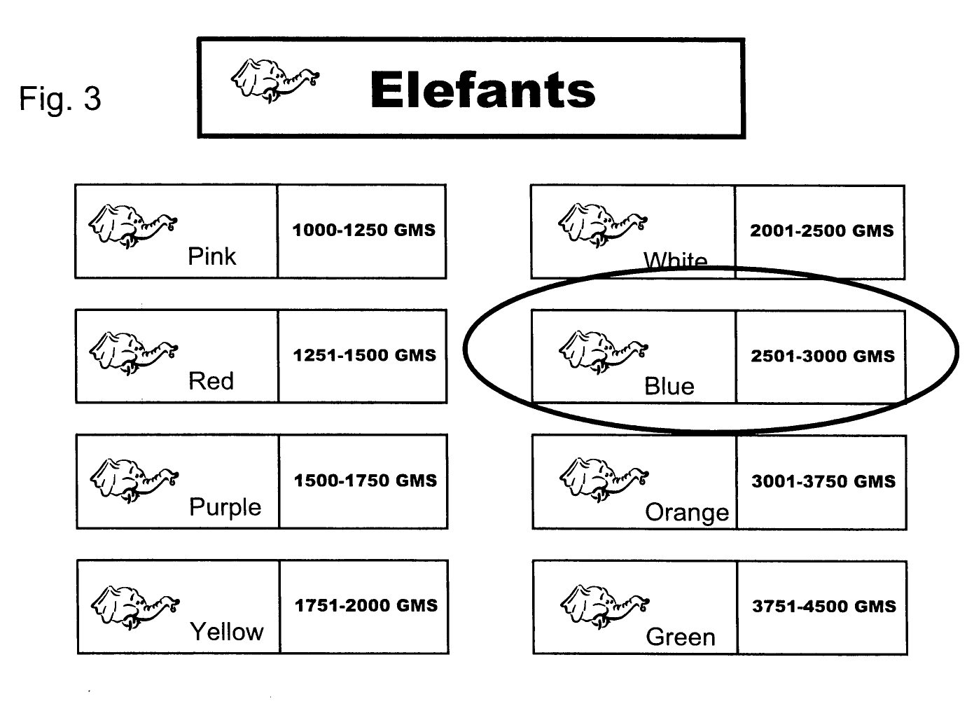

As shown in FIG. 3, the “Elefants” color categories correspond to respective weight ranges from between 1 kg (1000 gms) to 4.5 kg (4500 gms). An infant categorizes as “blue”, for example, will have a body weight of between 2501 and 3000 grams (2.5 to 3 kg).

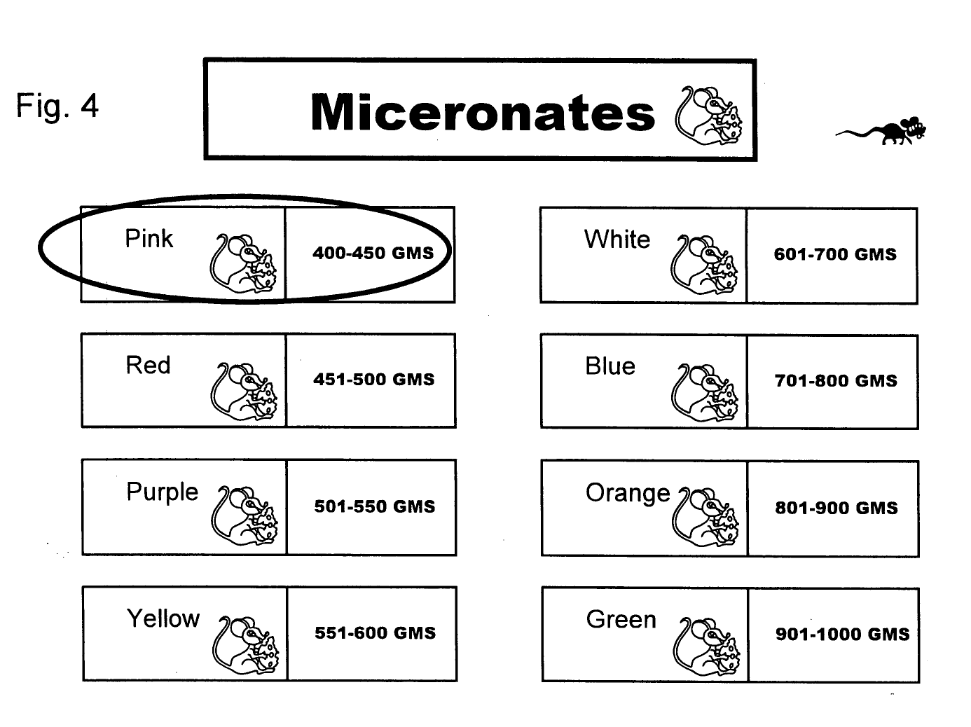

Similarly, as shown in FIG. 4, the Miceronates color categories correspond to respective weight ranges from between 400-450 gms to 901-1000 gms (1 kg).

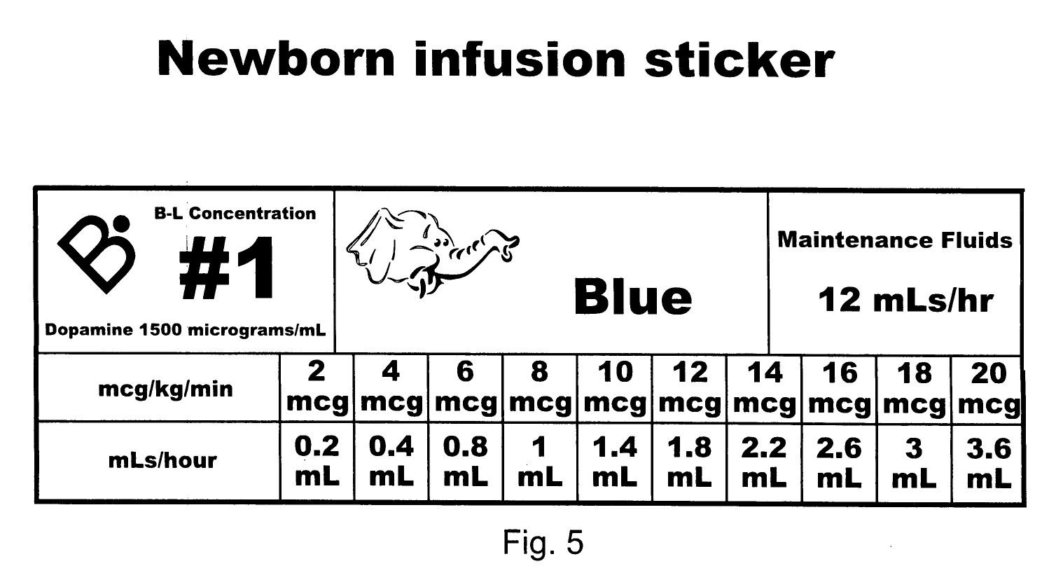

Referring now to FIG. 5, a drawing of a proposed Infants infusion sticker is shown. The information includes a prominent reference to a “Broselow-Luten” (B-L) # 1 Concentration, intended to be a “stock” concentration usable over a wide range of body weights. The B-L # 1 Concentration is, for example, equivalent to 1600 micrograms per milliliter (1600 mics/mL).

By reference back to FIG. 3, it can be determined that an infant weighing between 2501 and 3000 gms is a “blue elefant”. The sticker shown in FIG. 5 thus provides dosages of a standard (B-L #1) concentration of dopamine from 2-20 mcg/kg/min, as prescribed by a physician. These values provide the amount of the B-L # 1 Concentration to be administered per hour, in mLs. For example, a dosage of 10 mcg would require an infusion of 1.4 mL/hour of the B-L # 1 Concentration liquid. The sticker in FIG. 5 also provides for reference a standard Maintenance Fluid Value of 12 mL/hr.

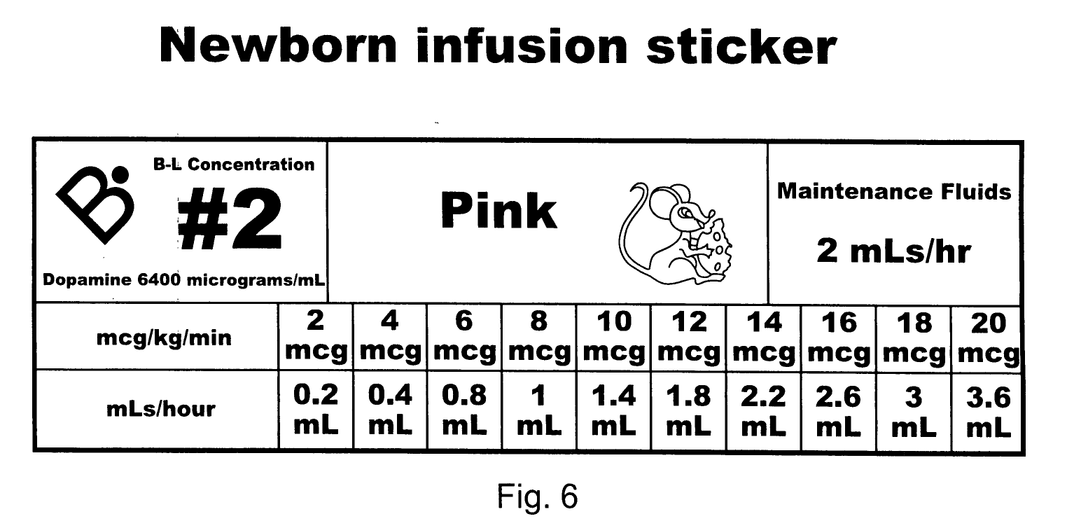

Referring now to FIG. 6, a drawing of another proposed Infants infusion sticker is shown. The information includes a prominent reference to a “Broselow-Luten” (B-L) # 2 Concentration, intended to be a “stock” concentration usable over a wide range of body weights within he neonatal body weight range. The B-L # 2 Concentration is, for example, equivalent to 6400 micrograms per milliliter (6400 mics/mL).

By reference back to FIG. 4, it can be determined that an infant weighing between 400-450 gram is a “pink micronate.” The sticker shown in FIG. 6 thus provides dosages of a standard (B-L #2) concentration of dopamine from 2-20 mcg/kg/min, as prescribed by a physician. These values provide the amount of the B-L # 2 Concentration to be administered per hour, in mLs. For example, a dosage of 10 mcg would require an infusion of 1.4 mL/hour of the B-L # 2 Concentration liquid. The sticker in FIG. 6 also provides for reference a standard Maintenance Fluid Value of 2 mL/hr, consistent with the very low body weight of the infant.

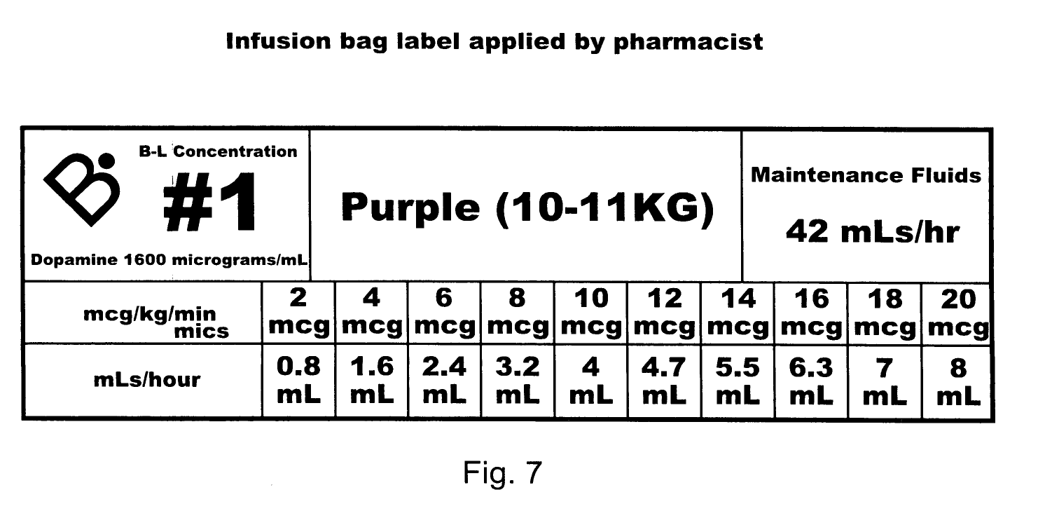

An example of an infusion bag label applied by a pharmacist to an infusion bag is shown in FIG. 7. The label indicates that the infusion is for a “Purple” pediatric patient weighing between 10 and 11 kg. Using a B-L Concentration # 1, a dopamine concentration of 1600 micrograms/mL, a physician prescribes a dosage ranging from 2-20 mcg, thus providing the nurse with the information that an infusion of from 0.8-8 ml/hr is required, with a total maintenance fluid requirement of 42 mLs/hr. In the examples described above, the likelihood of error is drastically reduced. The patient is clearly identified by color and weight. The concentration of the drug and the total maintenance fluid requirements are provided, as well. The total range of drug dosages is set out, immediately indicating to the nurse that an error will occur is a dosage outside the specified range occurs. The proper value is set on the IV machine and the proper dosage is administered by infusion.

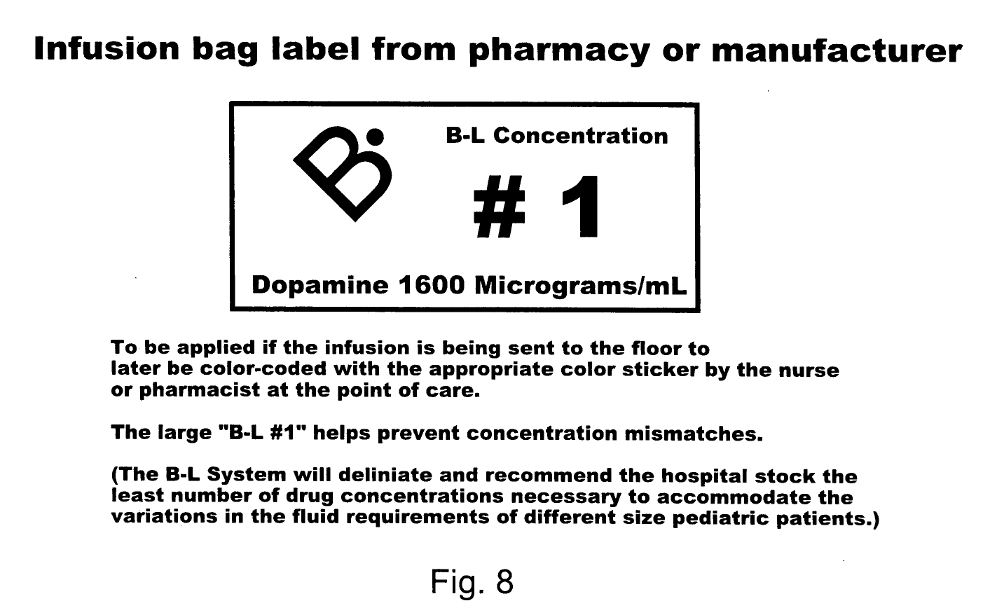

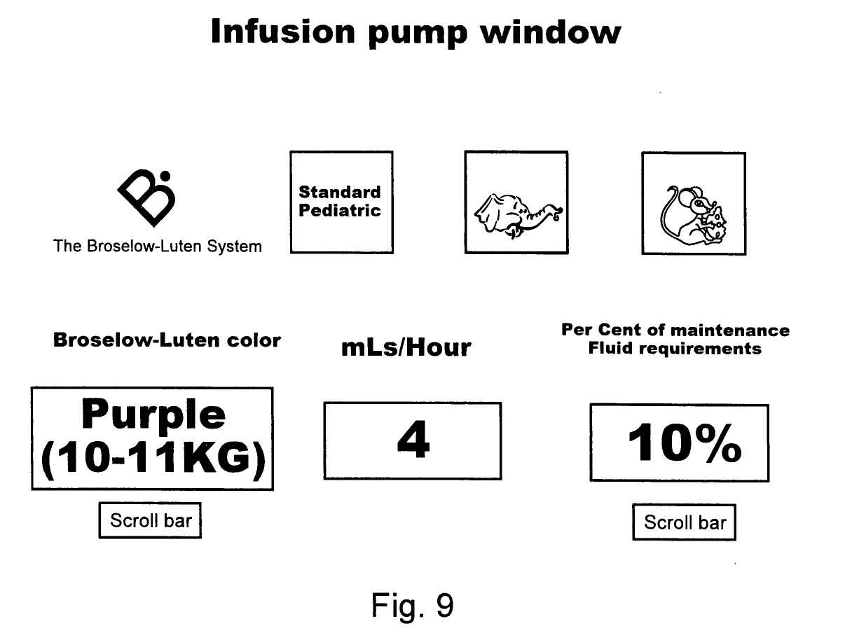

FIG. 8 represents an infusion bag label as received from a pharmacy or fluid manufacturer. A color-coded sticker corresponding to the weight of the patient would be attached once the bag was delivered to the patient's room for administration. The large reference to the “B-L # 1” prevents a concentration mismatch. Thus, a label is applied by the pharmacist responding to an order for “Dopamine for a Purple Child”, as shown in FIG. 7, or the pharmacist or drug manufacturer applies a label identifying the drug concentration by a large easily identifiable number representing the concentration of the drug as listed in the Broselow Luten System. By having this label already on the infusion, the nurse can safely label the drug at the bedside simply by matching the number on the medicine with the corresponding number on the infusion label.

In accordance with the invention pre-print sheets of labels are prepared that start from the small weight zone that are compatible with a given concentration to the largest size patient, including adults. The children's labels are indexed by color while the adult labels are indexed by a weight grouping such as 50 to 60 kgs. denoting a single label. If, for instance, the concentration of dopamine of 1600 micrograms/mL (B-L # 1) is available as a “stock” concentration, the manufacturer packages a single package insert that contains a series of labels that provide the microgram/mLs per hour grid allowing the user to select the appropriate label for that color or weight zone and apply it to the medicine bottle or infusion bag, thus customizing the instructions for a patient of that particular size.

Color can also be used to give instant access for child related medical information. For instance, a parent could carry a personal digital assistant (“PDA”) programmed with information useful for the age and weight of the child, with current information being downloaded periodically to correspond with the increasing weight of the child. For example, if a child began choking on a piece of food, activating the child's “color” and an icon for CPR or Choking on the PDA would activate a short segment of digital video footage showing the parent exactly how to do a “choking maneuver” on a child that size. Similarly, a health care provider using a color-coded dosing system could have a PDA with a screen giving doses of medications for Advanced Life Support while someone else performs CPR on a patient in front of them. To rapidly teach and reinforce proper technique an icon for CPR could be activated again showing in this instance accurate CPR technique for an infant exactly duplicating the size and indications for the infant actually in the resuscitation room.

Likewise, an infusion device giving IV Contrast dosing in the X-ray department of a hospital could have associated color-coded information and algorithms for treating an allergic reaction to the contrast as well as other emergency response information.

Another example would be on the computerized screen in a car designed for maps, travel information, etc. which could also incorporate an icon for proper passenger child restraint information, which along with the correct color-code could bring up a brief video segment demonstrating for the parent how properly to put a child in the correct child restraint and how to fit various child restraints into that particular model vehicle.

Another benefit of the color coded computer screen is to reassure the person using it that all calculations are indeed “for a blue child.” Since a computer is essentially a “black box” in which you input information and receive an answer, the color screen can serve as a real-life reassurance that the answer is correct and that the procedures are correct for a “blue” child. This would work not only for that operator, but because the color could be seen from a distance it could act as a “failsafe mechanism”for anyone in the room helping with a particular medical emergency or resuscitation recruiting them into a safety and error reduction team. This is a particular problem in many localities where non-native English speakers fill many professional and non-professional medical positions.

Similarly, for over-the-counter medications a series of color-coded spoons can be provided with the volume in mLs representing the correct volume for common matching OTC medications, such as acetaminophen. The spoons would be color-coded to the standard weights identified above. By filling the color-coded spoon the proper dose is automatically given.

A method of infusing a therapeutic fluid into a patient is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

I claim:

1. A method of infusing a therapeutic fluid into a patient, comprising the steps of:
(a) categorizing a universe of patient weights into a plurality of discrete weight ranges;
(b) correlating the weight ranges to a preselected series of identifiably distinct characteristics;
(c) determining an appropriate amount of a therapeutic substance for each of the plurality of weight ranges;
(d) determining normal maintenance fluid requirements each one of the plurality of weight ranges;
(e) adjusting the amount of administered maintenance fluid to compensate for the quantity of therapeutic fluid being administered;
(f) using the identifiably distinct characteristic correlated with the selected one of the plurality of weight ranges to visually correlate the patient with a proper therapeutic fluid dosage and a proper amount of maintenance fluid; and
(g) infusing the therapeutic fluid and maintenance fluid into the patient in amounts collectively comprising a proper amount of fluid based on the correlated weight range of the patient as indicated by the characteristic.

2. A method according to claim 1, wherein the preselected series of identifiably distinct characteristics comprises colors.

3. A method according to claim 2, wherein the step of categorizing patients comprises the further steps of categorizing the patients into pediatric, large and small infant neonatal subcategories, and further subcategorizing the infant and neonatal subcategories according to the series of identifiably distinct colors.

4. A method according to claim 2, wherein the step of using the identifiably distinct characteristic correlated with the selected one of the plurality of weight ranges to visually correlate the patient with a proper therapeutic fluid dosage and a proper amount of maintenance fluid comprises the step of providing a label for placement on an infusion bag, bottle, patient chart or patient record containing information selected from the group consisting of a designator identifying a concentration of a therapeutic composition, a color identifying one of the series of colors, an volume of maintenance fluids to be administered per unit of time, and the volume of therapeutic fluid to be administered per unit time.

5. The method according to claim 4, wherein the volume of therapeutic fluid to be administered per unit time is expressed in micrograms/kilogram/hour and milliliters/hour.

6. A method according to claim 1, 2, 3, 4 and 5, wherein the therapeutic fluid is selected from the group consisting of medications, electrolytes and nutrients.

7. A method according to claim 6, wherein the medication is dopamine.

8. A method according to claim 1, wherein the step of categorizing patients comprises the further steps of:
(a) categorizing the patients into pediatric, neonatal and adult subcategories;
(b) further subcategorizing the infant and neonatal subcategories according to the series of identifiably distinct colors; and
(c) further subcategorizing the adult subcategory into a plurality of discrete weight ranges.
